(19) United States
(12) Patent Application Publication
(10) Pub. No.: US 2012/0115211 A1
(43) Pub. Date: May 10, 2012

(54) Apparatus for Testing the Quality of a Fluid Sample

(75) Inventors: Tahmina Ajmal, Bristol (GB); Robert E.S. Bain, Bristol (GB); Stephen W. Gundry, Bath (GB); Philip Walsh, Bristol (GB); Craig I. Wightman, Bristol (GB)

(73) Assignee: The University of Bristol, Bristol (GB)

(21) Appl. No.: 13/266,685

(22) PCT Filed: Apr. 30, 2010
(86) PCT No.: PCT/GB10/50728

§ 371 Date: Jan. 10, 2012

(30) Foreign Application Priority Data
May 1, 2009 (GB) 0907605.0

(51) Int. Cl. C12M 1/34; G01N 33/00

(52) U.S. Cl. 435/287.1; 422/68.1

(57) ABSTRACT

Apparatus for testing the quality of a fluid sample comprising a body defining an interior space including a primary chamber and one or more secondary chambers, the apparatus having a first configuration in which it is arranged to hold at least some of the fluid sample in the primary chamber and a second configuration in which it is arranged to isolate a first portion of the fluid sample within the one or more secondary chambers whilst retaining a second portion of the fluid sample in the primary chamber.

The present invention relates to apparatus for testing the quality of a fluid sample, for example testing for the presence of impurities in a water sample. The present invention further relates to a system for testing the quality of a fluid sample.

As the Millennium Development Goals for water recognise, microbially contaminated drinking water is a major cause of diarrhoeal disease, responsible for the deaths of 1.8 million people every year (WHO, 2004) most of which are children in developing countries. In contrast, the development of new water testing technologies is driven by the needs of water companies in North America and Europe to adhere to the stringent standards set by regulatory authorities and, more recently, to concerns about bio-terrorism. Even with basic water testing equipment, skilled technicians and appropriate laboratory settings are rarely available in developing countries. As a result, there is a mismatch between the targets for technological development and the disease burden. This failure to develop appropriate diagnostics is analogous to the lack of investment by pharmaceutical companies to develop drugs to tackle diseases common only in developing countries.

When natural disasters occur, such as tsunami and earthquakes, agencies report that many of the attributable deaths are not the direct result of the disaster itself, but can be caused by subsequent outbreaks of disease, particularly from contaminated drinking water. Testing of drinking water sources after disasters presents particular problems due to the critical lack of staff, resources, and communications and transport infrastructure.

The World Health Organization issues Guidelines for Drinking-Water Quality. For bacteriological quality of drinking water, the WHO's Guidelines for drinking-water quality, state ‘(In) All water intended for drinking, E. coli or thermo tolerant coliform bacteria must not be detectable in any 100-ml sample’. Whilst adherence to this stringent standard is required and achieved by most developed countries, it is likely to be an unachievable target for most developing countries within the foreseeable future. This is particularly true where water is drawn from community sources in rural areas such as rivers or natural springs.

At present, many of the other available water testing technologies have been designed for use in developed countries. This is because the size of markets for water testing products is much greater in developed countries than in developing countries, where governments have only limited funds available for water testing. Many water testing technologies, such as the standard membrane filtration approach, require water samples to be collected in the field, stored under ice in transport containers, and then transported back to a microbiological laboratory. This microbiological laboratory needs to have appropriate facilities for testing samples, such as glassware incubators, lab benches, and facilities for the disposal of potentially hazardous waste, refrigerators, and trained technicians capable of undertaking water tests.

In remote areas of developing countries many of these facilities are simply unavailable. Ice for transporting water samples back to the laboratory may be impossible to obtain. The nearest microbiological laboratory may be a considerable distance away and there may be only very limited transport available for hard-pressed government environmental health technicians. Establishing a laboratory locally may also be difficult. Mains electricity may be either unavailable or available only sporadically and even buildings with workbenches and running water may be difficult to find. Many developing country organisations may be unable to afford the high consumables costs associated with some water tests. In many rural districts of developing countries, there is a lack of trained personnel able to carry out some of the more complex water testing procedures, such as calculating Most Probable Numbers of indicator bacteria or performing appropriate sample dilutions.

In recent years there has been some progress in the development of field kits for testing water. The University of Surrey developed the ‘DelAgua’ kit and this is still sold and used in the field, both in developing countries and by disaster relief agencies. It is based on the membrane filtration technique, requires a skilled technician and is time consuming. In more recent years, tests using Hydrogen Sulphide (H2S) have been developed to provide a simple ‘Presence/Absence’ result. An assessment of these tests (Sobsey and Pfaender, 2002) concluded (p37) ‘The H2S method in various modifications has been tested in many places in different waters and produced results reported as indicating it to be a reasonable approach for testing treated and untreated waters for faecal contamination. It offers advantages including low cost (estimated at 20% of the cost of coliform assays), simplicity and ease of application to environmental samples.’ However, the report noted several deficiencies in the reported assessments of the H2S test and commented ‘Because of these deficiencies, it is not possible to widely and unequivocally recommend H2S tests for the determination of faecal contamination in drinking water. There remain too many uncertainties about the reliability, specificity and sensitivity of the test for detecting faecal contamination of drinking water and its sources.’

Traditional laboratory tests include taking a 100 ml sample of water and passing it through a filter membrane. The residue left on the filter membrane is then cultured with staining reagents. After a period of incubation, the stained colonies are counted manually. In recent years, several manufacturers have produced reagents that use nutrient indicators to detect total coliforms and E. coli. Coliforms produce an enzyme that metabolises the nutrient indicators and cause either a change of colour or create fluorescence. These reagents are thus able to identify E. coli by visual or laser-based inspection. A known sample testing kit utilises one such nutrient indicator in conjunction with large sealable blister packs with a large number (50-97) of individual sample receiving wells. The nutrient indicator is mixed with a water sample which is then poured into the blister pack and the blister pack is subsequently sealed such that the individual wells are all filled with the sample and nutrient indicator mix. After an appropriate period of incubation the number of sample wells showing a positive result (indicative of contamination) is counted and statistical analysis applied to estimate the contamination level in MPN/100 ml. However, the sample and nutrient mixing, the filling of the blister pack, the counting of the positive results and the statistical analysis all require skilled or educated personnel which may not generally be available, for example, in developing countries.

The present applicant has developed a water test apparatus having a plurality of discrete chambers that are arranged to be filled with water to be tested. This apparatus is described in PCT/GB2006/004520. The chambers are individually dosed with reagent and growth media. Apparatus of this nature can be reasonably complex.

In other known water testing apparatus such as that disclosed in US 2005/0048597, a mixing chamber is provided in which a water sample is mixed with a reagent and growth media and subsequently directed into one or more holding chambers for incubation. The applicant has identified that this can lead to a device that is larger than is necessary. In some cases the use of known apparatus requires instructions to be followed, which can be unsuitable for unskilled users or those that cannot read or understand the instructions and/or functionality of the apparatus. Furthermore, another potential disadvantage with known apparatus is that, following incubation, a water sample may contain a high level of impurities, for example E. coli and waterborne pathogens. Such an incubated water sample therefore has the capability of damaging the health of a user or contaminating another water supply, should the incubated water sample be removed from the apparatus. Even if a user is trained to decontaminate a water sample, there exists a risk that the sample may be removed from the device before a decontaminant has been added or had enough time to take effect. This risk may be increased if the user is unskilled and does not fully understand the procedural steps that he or she should follow.

There is therefore a need for a method and apparatus for testing the quality of a fluid sample that substantially alleviates one or more of the above mentioned disadvantages.

According to a first aspect of the present invention, there is provided apparatus for testing the quality of a fluid sample comprising a body defining an interior space including a primary chamber and one or more secondary chambers, the apparatus having a first configuration in which it is arranged to hold at least some of the fluid sample in the primary chamber and a second configuration in which it is arranged to isolate a first portion of the fluid sample within the or each secondary chamber whilst retaining a second portion of the fluid sample in the primary chamber.

In some embodiments, at least some of the body may comprise a transparent or translucent material such that at least some of the internal space of the primary chamber and the one or more secondary chambers can be viewed from the exterior of the apparatus.

In some embodiments the body is generally cylindrical in shape and includes a column concentrically disposed with respect to the axis of the body, the column having a longitudinal duct arranged to remain in fluid communication with the primary chamber in both first and second configurations. At least some of the internal space of the secondary chambers and at least some of the internal space of the longitudinal duct within the column may be visible by viewing the apparatus from a fixed viewing angle. The column may include a longitudinally extending slot providing fluid communication between the longitudinal duct within the column and the primary chamber and the apparatus may further include a collar slidably surrounding at least a portion of the column, the collar being arranged such that, in use, irrespective of the orientation of the apparatus, at least some of the collar extends into the fluid sample. As will be appreciated by a skilled person, this can prevent a head of air within the primary chamber from entering the longitudinal duct when the apparatus is inverted. As will also be appreciated, for the collar to work in this way there must be at least a predefined volume of fluid within the apparatus.

In some embodiments the apparatus includes a sealing structure disposed within the interior space, wherein in the first configuration the sealing structure is arranged in a first position that permits fluid communication between the primary chamber and the one or more secondary chambers and in the second configuration the sealing structure is in a second position that inhibits fluid communication between the primary chamber and at least some of the one or more secondary chambers. The apparatus may include actuation means arranged to be movable relative to the body to move the sealing structure so as to change the apparatus between its first and second configurations and the actuation means may comprise a cam member arranged to rotate relative to the body and to engage with the sealing structure, the sealing structure being arranged such that it may rotate relative to the cam member but substantially not rotate relative to the body, the cam member comprising a cam surface arranged to bring about linear movement of the sealing structure in accordance with rotation of the cam member relative to the body.

In some embodiments, the central column includes a longitudinally extending slot providing fluid communication between the longitudinal duct within the column and the primary chamber and the sealing structure includes a collar slidably surrounding at least a portion of the column, the collar being arranged such that, in use, irrespective of the orientation of the apparatus, at least some of the collar extends into the fluid sample.

In some embodiments, the apparatus comprises a reagent containment means, wherein the apparatus has a third configuration wherein the reagent containment means is sealed from the internal space of the apparatus and a fourth configuration wherein the reagent containment means is in fluid communication with at least some of the internal space of the apparatus. Relative movement between first and second parts of the apparatus may be arranged to change the apparatus between its third and fourth configurations and the relative movement may be linear movement.

In some embodiments, the apparatus comprises decontaminant containment means, wherein the apparatus has a fifth configuration wherein the decontaminant containment means is sealed from the internal space of the apparatus and a sixth configuration wherein the decontaminant containment means is in fluid communication with at least some of the internal space of the apparatus. Relative movement between first and second parts of the apparatus may be arranged to change the apparatus between its fifth and sixth configurations and the relative movement may be rotational movement. One of the first and second parts may include a projection arranged to release decontaminant from the decontaminant containment means upon predefined movement of the first part relative to the second part.

In some embodiments, the apparatus comprises a closure member arranged to be sealingly coupled to the body to isolate the primary chamber from the exterior of the apparatus in a fluid tight manner. The closure member may be arranged to be coupled to the body such that it can move rotationally with respect thereto. Optionally or in addition, the closure member is arranged to be coupled to the body such that it can move linearly with respect thereto. The closure member may include an inlet, the closure member being arranged such that linear movement of the closure member relative to the body closes the inlet and purges excess fluid from the inner space. The apparatus may be arranged to be manipulated between configurations whilst the primary chamber is isolated from the exterior of the apparatus in a fluid tight manner by the closure member.

In some embodiments, the apparatus is arranged to permit manipulation thereof through a plurality of configurations in a predefined order and inhibit manipulation thereof through the plurality of configurations in any other order.

In accordance with a second aspect of the present invention, there is provided a system for testing the quality of a fluid sample including:

an electronic diagnostic device; and

apparatus for testing the quality of a fluid sample according to the first aspect,

the electronic diagnostic device being arranged to receive an input corresponding to the state of fluid contained within the plurality of chambers and generate an output indicative to the quality of the fluid sample, wherein the system includes a light source arranged to illuminate the chambers and the electronic diagnostic device is an optical reader arranged to measure the change in response of fluid contained in the chambers to generate the input.

In accordance with a third aspect of the present invention, there is provided the use of the primary chamber of apparatus according to the first aspect as both a chamber for receiving at least some of the fluid sample and testing at least some of the fluid sample.

Embodiments of the present invention will now be described, by way of non-limiting example only, with reference to the accompanying figures, in which:

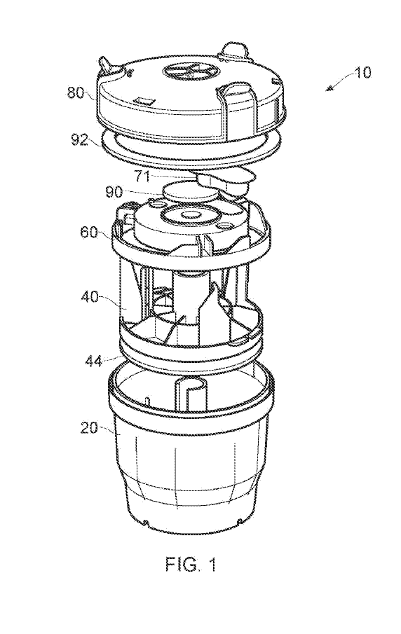

FIG. 1 is an exploded perspective view of apparatus according to an embodiment of the present invention;

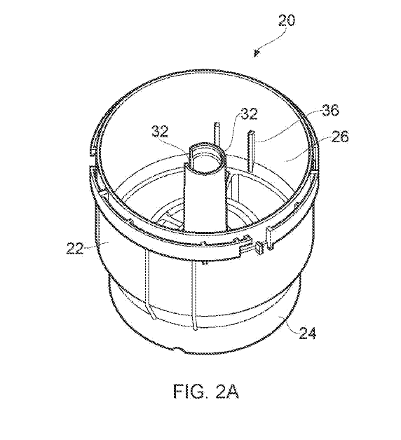

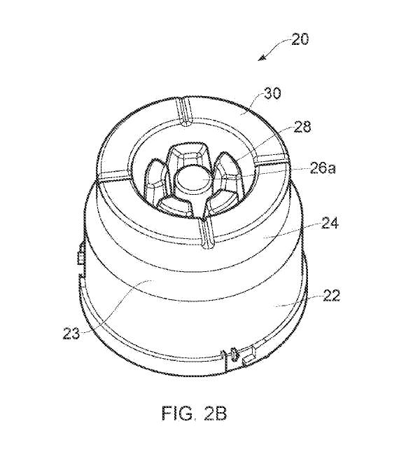

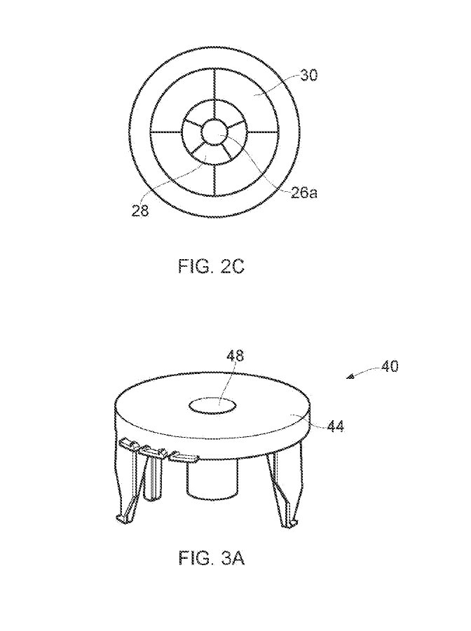

FIG. 2a is a perspective view of the top of the body of the apparatus of FIG. 1;

FIG. 2b is a perspective view of the base of the body of the apparatus of FIG. 1;

FIG. 2c is a plan view of the body of the apparatus of FIG. 1;

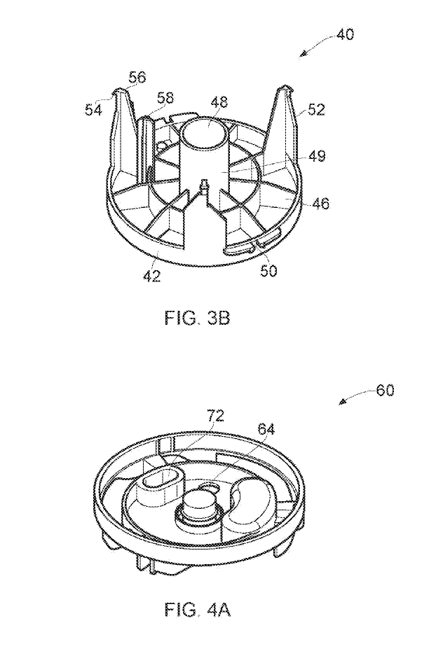

FIG. 3a is a perspective view of the bottom of the sealing structure of the apparatus of FIG. 1;

FIG. 3b is a perspective view of the top of the sealing structure of the apparatus of FIG. 1;

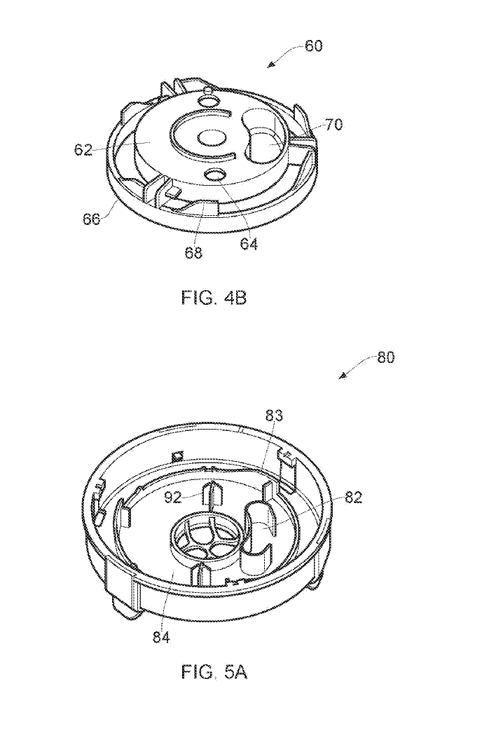

FIG. 4a is a perspective view of the bottom of the cam member of the apparatus of FIG. 1;

FIG. 4b is a perspective view of the top of the cam member of the apparatus of FIG. 1;

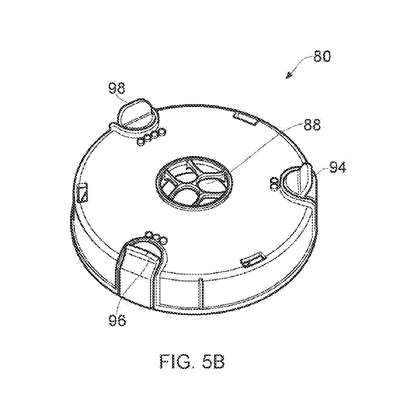

FIG. 5a is a perspective view of the bottom of the lid of the apparatus of FIG. 1;

FIG. 5b is a perspective view of the top of the lid of the apparatus of FIG. 1;

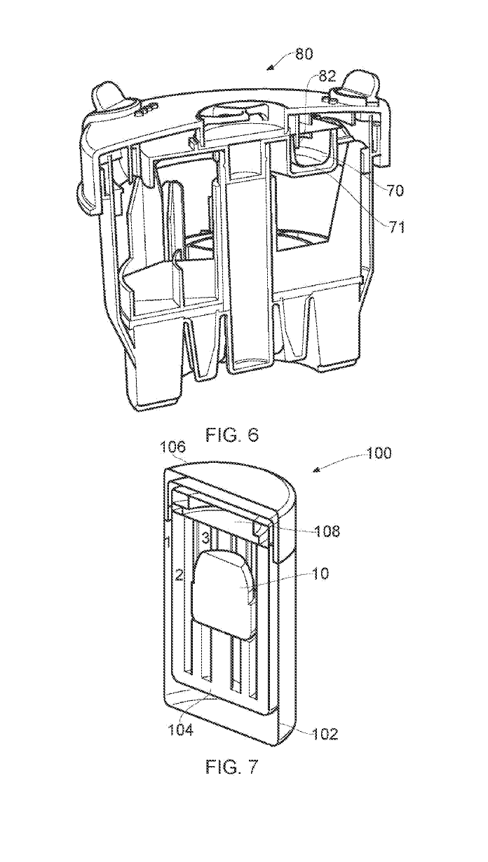

FIG. 6 is a cross section view through the axis of the apparatus of FIG. 1, when the apparatus is in a sealed configuration with the sub chambers isolated from the primary chamber; and

FIG. 7 is a perspective sectional view of an incubator according to an embodiment of the present invention.

Referring to FIG. 1, an exploded view of apparatus for testing the quality of a fluid sample, such as a water sample, is shown according to an embodiment of the invention. The apparatus comprises a number of parts that together enable the apparatus to be changed between a plurality of configurations. In the illustrated embodiment these include: a configuration wherein a primary chamber is in fluid communication with secondary chambers and a configuration wherein the secondary chambers are isolated from the primary chamber; a sealed and an unsealed configuration of the apparatus with respect to its outside environment; a configuration wherein reagent is isolated from a fluid sample in the inner volume of the apparatus and a configuration wherein reagent is released into the fluid sample; and a configuration wherein decontaminant is isolated from a fluid sample in the inner volume of the apparatus and a configuration wherein decontaminant is released into the fluid sample. The parts include a body 20, a sealing structure 40, a cam member 60 and a lid 80. Movement of one or more of these parts relative to another part brings about change between configurations, as described in detail below. Generally speaking, linear movement of the lid 80 relative to the body 20 changes between the sealed and unsealed configuration as well as releasing reagent. Rotation of the lid 80, and thus cam member 60, relative to the sealing structure 40 isolates and connects the primary and secondary chambers as well as releasing decontaminant.

FIGS. 2a and 2b show the body 20 of the apparatus. In the illustrated example the body 20 is unitary. However, in other embodiments the body 20 can be formed of two or more constituent body parts. The body 20 defines a plurality of chambers 26, 28, 30. The chambers 26, 28, 30 together define the interior space of the body 20. The chambers 26, 28, 30 are, in one configuration of the apparatus, in fluid communication with one another. The apparatus further includes a sealing structure 40 which, in another configuration of the apparatus, isolates a chamber 26, 28, 30 from another chamber 26, 28, 30. The body 20 is formed of a translucent material so that at least some of the chamber-space of each chamber 26, 28, 30 can be viewed from the outside of the device. However, in some embodiments only a particular area, namely the “viewing portion”, may comprise a transparent or translucent material. In some embodiments the body 20 is constructed of an ultraviolet transmitting material.

In the illustrated example, the body 20 defines a primary chamber 26 and a plurality of sub chambers 28, 30. The volume of the primary chamber 26 is such it can hold a capacity of 100 ml of water. There are four medium sized sub chambers 30 and five small sub chambers 28. A medium sub chamber 30 has a capacity of 7 ml of water and a small sub chamber 28 has a capacity of 0.7 ml of water. However, it is to be understood that the body 20 may, in other embodiments, have two or more chambers 26, 28, 30 of any desirable capacity, such as a plurality of substantially identically configured sub chambers. In some embodiments the body defines a larger number of smaller chambers, for example twenty. The smaller chambers may be of differing sizes or in groups of differing sizes. These smaller chambers mean that the apparatus can have a higher range, as a number of smaller chambers can generally detect higher concentrations of a contaminant in a fluid sample than a number of larger ones, and a larger number of chambers providing better precision. However, providing a large number of small chambers can make interpreting the results more complicated. To assist a user in visually interpreting the results the sub chambers may be arranged in clusters such that a first cluster can be visually distinguished from other clusters and a user can, for example, count a cluster of chambers as a single entity if each chamber within the cluster displays a colour change.

The sub chambers 28, 30 are provided towards the closed base of the body 20. Each sub chamber 28, 30 has an opening through which it is in fluid communication with the primary chamber 26. Each sub chamber 28, 30 opens onto a common plane such that they together define a contact surface which may be sealed by a planar face of a sealing structure. The common plane of the contact surface will be referred to as the “sealing plane”. Of course, other suitable configurations will be apparent to a skilled person.

The sub chambers 28, 30 are arranged to be viewable from a fixed viewing angle. In the illustrated example, the sub chambers 28, 30 are arranged such that at least some of the internal space of each sub chamber 28, 20 is viewable from the base. The base therefore forms a viewing portion. The primary chamber 26 includes a primary chamber well 26a that is viewable from the viewing portion, as described below. In preferred embodiments the entirety of the internal space of each sub 28, 30 chamber is viewable from the base. Thus, when a portion of a fluid sample is present within a sub chamber 28, 30 it will be viewable from the exterior of the apparatus 10. In some embodiments the chambers 26, 28, 30 are arranged to be read by an optical viewing device, as described in more detail below.

A column 32 extends from the opening of the primary chamber well 26a into the first cylinder 22 and terminates at a free end. The column 32 has a generally circular cross-section and is hollow such that the column 32 defines a generally cylindrical duct that extends longitudinally from one end of the column 32 to the other. A slot 34 is formed through the column 32 wall and extends longitudinally from the free end to the sealing plane. The column 32 is advantageous as it provides a surface about which other parts of the apparatus can rotate or be mounted, as described in more detail below. The column 32 also enables water within the primary chamber 26 to be viewed from the base of the body 20, as described in more detail below.

Referring to FIGS. 3a and 3b, the sealing structure 40 of the apparatus 10 of FIG. 1 is shown. The sealing structure 40 is movable between a first position where the sub chambers 28, 30 are each in fluid communication with the primary chamber 26, and a second position where one or more of the sub chambers 28, 30 are isolated from the primary chamber 26. In the illustrated example the sealing structure 40 comprises a body plate 42 which is generally circular in shape and arranged to isolate each of the sub chambers 28, 30 from the primary chamber, as well as from each other. The body plate 42 is provided with a planar seal 44 on its “sealing face” that can be deformed by the contact surface of the body 20 to create a fluid-tight seal there between. The sealing structure 40 has a central bore 48 arranged to receive the column 32 of the body 20. It will therefore be appreciated that the sealing structure 40 can slide along the column 32 in a first direction away from the base, or a second direction towards the base. A collar 49 extends from the rear face of the sealing structure 40. The collar 49 is concentric with respect to the bore 48. Rotation of the sealing structure 40 relative to the column 32 is inhibited by guide rails 36 (See FIG. 2A) extending along the inside wall of the first cylinder 22, parallel to the column 32, which are received by guide slots 50 of the sealing structure 40. However, any suitable means may be provided to inhibit the sealing structure 40 from rotating relative to the body 20. Furthermore, it will be appreciated that in other embodiments the sealing structure 40 may be actuated in a different manner, which may include rotation relative to the body 20 such as would be the case if the sealing structure 40 were to include a threaded inner bore rotatably coupled to a threaded column.

The sealing structure 40 includes a plurality of arms 52 projecting from its rear face, each arm 52 having a hook 54 provided at the free end thereof. The arms 52 are arranged to receive a force applied to the head portions 56 to press the sealing structure 40 against the contact surface of the body 20 at the sealing plane. The hooks 54 are arranged to interface with a cam member 60 to enable the sealing structure 40 to be drawn away from the sealing plane. Actuation of the sealing structure 40 is described in more detail below. A decontaminant piercing member 58 extends from the rear face of the sealing structure. The decontaminant piercing member 58 is arranged to pierce decontaminant retaining means when the apparatus is in a specific configuration, as described below. It is however to be understood that, when the apparatus is arranged to contain a decontaminant, any suitable means may be used to release the decontaminant upon the apparatus entering the relevant configuration.

Referring to FIGS. 4a and 4b, the cam member 60 of the apparatus 10 of FIG. 1 is shown. The cam member 60 in the illustrated example comprises a plate body 62 arranged to be supported on the free end of the column 32 such that the cam member 60 can rotate about the column 32 in a fixed plane. The plate body 62 has a pair of engagement apertures 64 via which a lid can engage the cam member 60 as described below. A peripheral rail 66 is connected to the plate body 62 and has an axial cam surface 68 which faces away from the sealing structure 40, as the latter is positioned in use and along which the hooks 54 of the arms 52 are arranged to travel. It will be appreciated that due to the cam member 60 being mounted on the column 32, the axial cam surface 68 of the peripheral rail 66 can bring about movement of the sealing structure 40 towards and/or away from the cam member 60 upon relative rotation between the sealing structure 40 and cam member 60. The configuration of the cam surface 68 is described in more detail below in relation to the apparatus in use.

In the illustrated example the plate body 62 of the cam member 60 includes a reagent station 70 that is bounded by a peripheral sidewall extending towards the sealing structure 40, as the latter is positioned in use. Reagent and growth media may be retained in the reagent station 70 by any suitable means, for example being held in a blister pack 71 or other moisture-resistant packaging. Whilst the apparatus is in a filling configuration, in which fluid may be introduced to the interior space, the reagent and growth media are not in fluid communication with the interior space of the apparatus 10. When the apparatus is changed to a sealed configuration, in which mixing may occur, the reagent and growth media are permitted to be in fluid communication with the interior space. In the illustrated example this is enabled by a reagent piercing member 82 provided on the lid 80, as described below. However, in other embodiments the reagent and growth media may be released by any suitable means. In some embodiments a fluorogenic reagent may be used, such as 4-Methylumbelliferyl-β-D-glucuronide (MUG). However, it should be noted that any reagent may be used that is suitable for testing the quality of a fluid sample, for example a chromogenic reagent such as 5-bromo-4-chloro-3-indolyl-β-D-glucuronide (X-gluc). In some embodiments one or more functional agents or additives may be provided along with the reagent, for example a growth media including carbon sources such as yeast extract or peptone, one or more salts or buffers to maintain a pH close to neutral, an additive such as sodium dodecyl sulphate to suppress the growth of gram-positive bacteria, or an additive such as sodium thiosulfate to neutralise any chlorine residual. Whilst in the illustrated example the apparatus is arranged to contain a reagent, and in some cases functional agents or additives, that is mixed with the water sample whilst the apparatus is sealed, in other embodiments the apparatus may be arranged to receive a water sample that has been pre-mixed with reagent and in some cases growth media.

The plate body 62 of the cam member 60 in the illustrated example includes a decontaminant station 72. A decontaminant such as chlorine may be retained in the decontaminant station 72 by, for example, foil or other moisture-resistant packaging. However, where the apparatus is arranged to contain a decontaminant, any suitable decontaminant retaining means may be provided. When the apparatus is in any of the filling to the incubating configurations, decontaminant is prevented from being in fluid communication with the interior space by the retention means. When the apparatus is changed to a decontaminating configuration, decontaminant is released into the interior space. In the illustrated example, this is achieved by rotating the cam member 60 relative to the sealing structure 40 to a position where the piercing projection 58 that extends from the non-sealing face of the sealing structure 40 breaks the foil packaging. However other means of releasing the decontaminant will be apparent. Although the apparatus 10 in the illustrated example is arranged to contain a decontaminant, in other embodiments the apparatus may not contain a decontaminant.

Referring to FIGS. 5a and 5b, the lid 80 of the apparatus 10 of FIG. 1 is shown. The lid 80 is arranged to be coupled to the body 20 so as to seal the interior space from the exterior of the apparatus 10. In the illustrated example the lid 10 comprises generally circular lid plate 84, having an outer face which, in normal use, faces away from the body 20 and an inner face which, in normal use, faces the body 20. A peripheral rim 86 extends from the inner face and is arranged to receive the free end of the first cylinder 22 of the body 20. The lid plate 84 includes an inlet 88 through which fluid may pass. The inlet 88 includes a grating to inhibit ingress of gross solids when introducing a test sample. A seal 93 is provided to create a fluid tight seal between the lid 80 and the body 20 when the apparatus is changed to its sealed configuration. A seal 90 is provided on the cam member 60 at a location corresponding to the inlet such that the inlet can be sealed by moving the cam member 60 into contact with the seal 90. However, other means of sealing will be apparent, such as providing a seal around the inlet 88 on the interior face of the lid. Engagement members 92, corresponding to the engagement apertures 64 of the cam member 60, extend from the inner face of the lid plate 84. A reagent piercing member 82 extends from the inner face of the lid plate. The reagent piercing member 82 comprises a skirt, corresponding in shape to the reagent station 70. The broken part of the skirt is to assist fluid inside the primary chamber 26 mixing with reagent contained within a blister pack in the reagent station 70. A plurality of cam ridges 83 are provided on the inside face of the lid 80. The cam ridges 83 are arranged to act against the arms of the sealing structure 40 to force the sealing structure 40 to seal the sub chambers 28, 30 as described below.

The free end of the first cylinder 22 of the body 20 is arranged to cooperate with the lid 80 such that the lid 20 may be moved between a first position, where the lid 20 is maintained at a position spaced from the body 20, and a second position wherein the interior space is sealed by the lid 20. When in the first position, fluid, for example water, may enter at least some of the primary chamber 26 via the inlet of the lid. The engagement members 92 are spaced from the engagement apertures 64 and the reagent piercing member 82 is spaced from the reagent station 70. Once a quantity of fluid has entered the primary chamber 26 the lid 80 may be moved to the second position. This is achieved by axially moving the lid 80 relative to the body 20.

In use, with the lid 80 in the first position such that the apparatus is in a filling configuration, water to be tested is caused to enter the apparatus through the inlet. Once a predetermined quantity of water is contained in the interior space, for example once the primary chamber 26 is at capacity or once the interior space is at capacity, the lid 80 is moved to the second position. This puts the apparatus into a sealed configuration, as shown in FIG. 6 and, in doing so, a number of actions occur. Firstly, the inlet 88 is sealed so as to prevent fluid from passing through it. Excess water residing in the lid cavity defined between the peripheral lip 86 may be expelled through the inlet as the lid 80 moves from the first to the second position. Secondly, the reagent piercing member 82 enters the reagent station 70 and punctures the foil blister pack 71 containing reagent. The reagent and growth media is then free to mix with water within the apparatus 10. Thirdly, the engagement members 92 move into the engagement apertures 64, such that subsequent rotation of the lid 80 will bring about corresponding rotation of the cam member 60. In the sealed configuration the apparatus 10 is arranged to inhibit fluid from entering the internal space from the exterior of the apparatus 10 and vice versa. With the apparatus 10 in a sealed configuration, the apparatus 10 may be shaken for a time duration that is suitable to ensure that most, if not all, of the reagent and growth media have mixed with the water sample. A typical time is 3 minutes. It should be noted that a user is able to see into the primary chamber 26 by way of the primary chamber well 26a disposed at the base of the hollow column 32 because the primary chamber well 26a is in fluid communication with and forms a part of the primary chamber 26. Consequently a user can see the water sample by viewing the apparatus 10.

The apparatus 10 according to this embodiment is arranged to maintain the internal space sealed from the environment external to the apparatus 10 throughout all further stages. This is achieved by the lid 80 and body 20 together being arranged to inhibit the lid 80 moving from the second position back to the first position. However, any suitable means for preventing fluid inside the apparatus from escaping may be provided. It will be appreciated that the purpose of this feature is to inhibit potentially dangerous fluid samples from being withdrawn from the apparatus. In some embodiments, however, the apparatus is arranged to permit a fluid sample to be removed after testing, thereby making the apparatus re-usable. In some embodiments where the apparatus is arranged to be reusable, external reagent, growth media and/or decontaminant may be used i.e. apparatus does not include a reagent and growth media and/or decontaminant station and supporting release mechanism. The reagent and growth media may be added to the fluid sample before the apparatus is put into a sealed configuration. The decontaminant may be added via a set of complimentary opening doors such that an inner door is closed while the outer is open and vice versa, to allow the decontaminant to be introduced to the incubated fluid sample without a high likelihood that the incubated fluid sample can escape to the exterior of the apparatus. Where the apparatus is arranged to be re-usable, it may require sterilisation, either in the field or in regional sterilisation centres and may be constructed of a durable material such as glass or engineering plastic. Some possible examples of methods of sterilisation in field are: disinfection with chlorine and quenching sodium thiosulfate; burning of ethanol; and solar disinfection.

The next stage is putting the apparatus 10 into an incubation configuration. In the illustrated example the apparatus 10 is arranged for the water sample to be poured into the primary chamber 26 whilst the sub chambers 28, 30 are sealed by the sealing structure 40. The mixing of the water sample with the reagent and growth media therefore occurs in the primary chamber 26. In order to put the device into an incubating configuration, the user opens or breaks off a catch 94 on the lid 80 that is, in its normal state, arranged to inhibit rotation of the lid 80 relative to the base. The lid 80 may then be rotated in a predefined direction. As noted above, rotation of the lid 80 brings about corresponding rotation of the cam member 60, due to the engagement members engaging with the cam member 60 via the engagement apertures. Rotation of the cam member 60 causes the sealing structure 40 to move along the column 32 towards the cam member 60, due to the configuration of the cam surface 68. As the sealing surface of the sealing structure 40 moves away from the contact surface of the sub chambers 28, 30, a portion of the water sample and reagent/growth media solution can enter the sub chambers 28, 30. It should be noted that in some embodiments the reagent may be released by rotational movement between two parts of the apparatus, as will be apparent to a skilled person.

Further rotation of the lid 80 is inhibited when the seal is at its furthest location from the sealing surface by a second catch 96 on the lid 80. This may prompt the user to check to see that the sub chambers 28, 30 are each full to capacity. The second catch 96 may then be opened or snapped off to allow further rotation of the lid 80 in the predetermined direction relative to the base. This stage of rotation causes the cam ridges 83 on the lid 80 to come into contact with the heads 58 of the arms 52, which transmits a compressive force axially against the arms 52. This compressive force moves the sealing structure 40 back along the column 32 towards the sub chambers 28, 30 and into its second position wherein the sub chambers 28, 30 are isolated from the primary chamber 26 by the sealing structure 40. It should be noted that in the illustrated example, rotation of the lid 80 relative to the body 20 in the opposite direction to the predetermined direction is inhibited by a resilient detent acting upon a toothed profile extending around the inner circumference of the lid, so as to permit rotation in one direction but inhibit rotation in the opposite direction. This mechanism inhibits a user from reversing the apparatus process and alternative mechanisms will be apparent. However, in other embodiments the apparatus 10 may be arranged such that the functions can be reversed. With a portion of the fluid sample sealed in the sub chambers 28, 30 and a second portion within the primary chamber, the apparatus is in an incubating configuration.

In another embodiment of the present invention, the apparatus is arranged for the water sample to be poured into the primary chamber 26 whilst the sub chambers 28, 30 are in fluid communication with the primary chamber 26 i.e. the sealing member is not isolating the sub chambers 28, 30 from the primary chamber. The mixing of the water with the reagent and growth media therefore occurs in the entirety of the inner space, except of course for the space occupied by the internal components of the apparatus such as the sealing structure 40 and cam member 60. The apparatus may then be put into its incubating configuration as described above with reference to the illustrated example. It should be noted that when the apparatus is arranged for the water sample to be poured into the primary chamber 26 whilst the sub chambers 28, 30 are in fluid communication with the primary chamber, it removes the requirement for the initial step of unsealing the sub chambers 28, 30 to permit the filling thereof. Consequently, the apparatus in such a case may not require rotation inhibition means to oppose rotation of the lid 80 to bring about the initial step of unsealing the sub chambers 28, 30, which may in some embodiments simplify the apparatus. It will also be appreciated that the cam member 60 may be simplified.

Once the apparatus is in its incubating configuration it may be incubated for a period of time, within a fixed temperature range, to allow impurities, such as E. coli strains, to grow to a level suitable for detection. It will be appreciated that with the base of the apparatus 10 facing downwards, the primary chamber 26 will contain a head of air that has left the sub chambers 28, 30 when they have been filled with a portion of the water sample. The collar 49 that extends from the rear face of the sealing structure 40 is arranged to be long enough such that when the apparatus is inverted, i.e. the base is facing upwards, the collar 49 extends through the head of air and into the portion of the water sample in the primary chamber 26. Consequently, this enables the portion of the water sample located within the primary chamber well 26a to remain there when the apparatus is inverted, as may be the case during incubation. The collar 49 thus inhibits bubbles from entering the window 26a into the primary chamber 26. In the illustrated example the apparatus is arranged to be placed in an incubator such as that shown in FIG. 7. The incubator 100 comprises a vacuum flask 102 having an inner lining 104 comprising phase change material (PCM). The incubator 100 may be filled with hot water which transfers heat energy to the PCM. The PCM melts and continues to store heat energy from the hot water primarily in the form of latent heat. The hot water may be emptied from the incubator 100 and the apparatus placed in the incubator 100 with the lid 80 facing down i.e. the lid 80 facing the base of the incubator. An incubator lid 106 may be close to seal the incubator. The PCM solidifies and releases its stored energy as heat at a generally constant temperature. This configuration is independent of an electrical supply. The type of PCM is chosen to maintain the interior of the incubator at a consistent temperature suitable for incubating the water sample. The lid of the incubator may or may not contain a viewing port 108 though which the base of the apparatus can be viewed. The viewing port enables a person to look at the apparatus or the apparatus to be viewed by an optical viewing device or image capture device. The incubator 100 may also include a light source, for example an excitation light source which can be used to show the presence of impurities when a fluorogenic reagent is used. If the fluorogenic reagent is MUG the excitation light source will be ultraviolet (UV). It will be appreciated that an excitation light source will generally not be provided if a chromogenic reagent is used. Providing consistent lighting on the surface of the apparatus 10 may make it easier to read results whether by eye or using a reader, such as a mobile phone or an optical reader.

Once the water sample that has been divided amongst the primary chamber 26 and sub chambers 28, 30 has been viewed, in some embodiments the apparatus may be changed to a decontamination configuration. Referring to the illustrated example, this may be achieved by opening or breaking off a third clip 98 on the lid 80. The third clip 98 is, when in its closed configuration, arranged to inhibit rotation of the lid 80 relative to the body 20 between a position where the apparatus is in the incubation configuration and a position where the apparatus is in the decontamination configuration. Opening or breaking off the third clip permits the lid 80 to rotate in a predefined direction relative to the body 20. Such rotation causes the cam member 60 to rotate as described above. The configuration of the cam is such that rotation during this stage unseals the sub chambers 28, 30 by withdrawing the sealing structure 40 from the sealing plane. Furthermore, the decontaminant piercing member 58 comes into contact with and pierces the decontaminant retaining means, so as to release the decontaminant into the inner space. Consequently, the entirety of the water sample, i.e. both the portion stored in the primary chamber 26 and portion stored in the sub chambers 28, 30, is in fluid communication with the decontaminant substance. In the illustrated example the apparatus is configured to inhibit any further change of configuration. However, as noted above, in some embodiments the apparatus may permit a user to manipulate the apparatus to one or more of the previous configurations or one or more further configurations so as to allow the water sample to be expelled and the apparatus be reused. In some embodiments the apparatus may require disassembly with a special tool. Furthermore, it should be noted that in some embodiments the decontaminant may be released by linear movement between two parts of the apparatus, as will be apparent to a skilled person.

The illustrated apparatus 10 therefore provides a self contained device that can be indexed through a predefined sequence of functions whilst remaining sealed. As a result, a user is not exposed to the microbiological pathogens and other organisms which may be present in large numbers in the incubator sample. This is advantageous as it enables an unskilled worker to test a fluid sample in a simple safe manner and minimises the risk of contamination of the sample. The apparatus therefore requires little or no training and no microbiological training is required. Other than an incubation environment, the apparatus does not require lab equipment such as glassware or an autoclave and does not require resources such as distilled water or electricity. There are only a few simple steps to the testing process. The user interface is arranged to discourage misuse, for example the apparatus inhibits the completion of all the steps in a single twist or to go back a step. Furthermore, the apparatus is space efficient due to the fact that at least a portion of the primary chamber, which is used for mixing, is also used for incubating and viewing the test sample. As will be appreciated from the foregoing description, a test chamber is a chamber in which testing of a fluid sample occurs, for example the primary chamber being arranged such that the sample isolated therein can be incubated and results taken by viewing the incubated sample. This provides for a smaller overall volume apparatus than would be required if the primary chamber was used for mixing only. The various parts of the apparatus are suitable for injection moulding. One or more of these advantages also applies to the other embodiments of the present invention, as appropriate.

In the present example the exterior of the apparatus includes visual markings (not shown) to clearly define the individual test stages to a user of the apparatus. This is an optional feature that can improve the ease of use of the apparatus.

In some embodiments, an electronic device may be used to interpret the results of the apparatus 10. An optical reader may be used to read the results of a test. An optical reader may be capable of reducing the time to result, especially in heavily contaminated waters where it is known that detectable levels of growth occur at an earlier time. An optical reader in one embodiment comprises a plurality of light sources and detectors arranged with one pair for each chamber 26, 28, 30 of the apparatus 10. In other embodiments there may be a different fixed number of light sources and a detector per chamber or different numbers of detectors. To interpret the signals from each detector, electronic circuits are arranged to calculate the number of positive chambers of each size and/or the total number that are positive and display these. An advantage of using an optical reader is that a complex array of many chambers or different sizes of chamber could be interpreted quickly and accurately. Furthermore, the fluid sample within some chambers may be weakly fluorescent or coloured at the end of the test and in such a case the use of an optical reader can produce repeatable results and may ensure that the user need not make a difficult judgment. An optical reader may thus provide reliable reading of the results and unambiguous read out for the user and is potentially more reliable than a mobile phone reader and may provide quicker results.

Although the appended claims refer to numbered configurations of the apparatus, it is to be understood that the apparatus need not be arranged such that the configurations occur in the numbered sequence and may occur in any suitable order. Furthermore, in some embodiments, a single actuation may change the apparatus between more than two configurations, for example, pressing the lid to both seal the apparatus from the outside environment and puncture a reagent blister pack.

1. Apparatus for testing the quality of a fluid sample comprising a body defining an interior space including a primary chamber and one or more secondary chambers, the apparatus having a first configuration in which it is arranged to hold at least some of the fluid sample in the primary chamber and a second configuration in which it is arranged to isolate a first portion of the fluid sample within the one or more secondary chambers whilst retaining a second portion of the fluid sample in the primary chamber.

2. Apparatus according to claim 1, wherein the primary chamber is arranged as a test chamber.

3. Apparatus according to claim 1, wherein at least some of the body comprises a transparent or translucent material such that at least some of the internal space of the primary chamber and the one or more secondary chambers can be viewed from the exterior of the apparatus.

4. Apparatus according to claim 1, wherein the body is generally cylindrical in shape and includes a column centrally disposed with respect to the axis of the body, the column having a longitudinal duct arranged to remain in fluid communication with the primary chamber in both first and second configurations.

5. Apparatus according to claim 4, wherein at least some of the internal space of the secondary chambers and at least some of the internal space of the longitudinal duct within the column is visible by viewing the apparatus from a fixed viewing angle.

6. Apparatus according to claim 4, wherein the column includes a longitudinally extending slot providing fluid communication between the longitudinal duct within the column and the primary chamber and the apparatus further includes a collar slidably surrounding at least a portion of the column, the collar being arranged such that, in use, irrespective of the orientation of the apparatus, at least some of the collar extends into the fluid sample.

7. Apparatus according to claim 1, further comprising a sealing structure disposed within the interior space, wherein in the first configuration the sealing structure is arranged in a first position that permits fluid communication between the primary chamber and the one or more secondary chambers and in the second configuration the sealing structure is in a second position that inhibits fluid communication between the primary chamber and at least some of the one or more secondary chambers.

8. Apparatus according to claim 7, further comprising actuation means arranged to be movable relative to the body to move the sealing structure so as to change the apparatus between its first and second configurations.

9. Apparatus according to claim 8, wherein the actuation means comprises a cam member arranged to rotate relative to the body and to engage with the sealing structure, the sealing structure being arranged such that it may rotate relative to the cam member but substantially not rotate relative to the body, the cam member comprising a cam surface arranged to bring about linear movement of the sealing structure in accordance with rotation of the cam member relative to the body.

10. Apparatus according to claim 1, comprising reagent containment means, wherein the apparatus has a third configuration wherein the reagent containment means is sealed from the interior space of the apparatus and a fourth configuration wherein the reagent containment means is in fluid communication with at least some of the internal space of the apparatus and optionally wherein relative movement between first and second parts of the apparatus is arranged to change the apparatus between its third and fourth configurations and optionally wherein the relative movement is linear movement.

11. Apparatus according to claim 1, further comprising decontaminant containment means, wherein the apparatus has a fifth configuration wherein the decontaminant containment means is sealed from the internal space of the apparatus and a sixth configuration wherein the decontaminant containment means is in fluid communication with at least some of the internal space of the apparatus and optionally wherein relative movement between first and second parts of the apparatus is arranged to change the apparatus between its fifth and sixth configurations and optionally wherein the relative movement is rotational movement and optionally wherein one of the first and second parts includes a projection arranged to release decontaminant from the decontaminant containment means upon predefined movement of the first part relative to the second part.

12. Apparatus according to claim 1, further comprising a closure member arranged to be sealingly coupled to the body to isolate the interior space from the exterior of the apparatus in a fluid tight manner.

13. Apparatus according to claim 12, wherein the closure member is arranged to be coupled to the body such that it can move rotationally with respect thereto.

14. Apparatus according to claim 12, wherein the closure member is arranged to be coupled to the body such that it can move linearly with respect thereto.

15. Apparatus according to claim 12, wherein the closure member includes an inlet, the closure member being arranged such that linear movement of the closure member relative to the body closes the inlet and purges excess fluid from the inner space.

16. Apparatus according to claim 12, wherein the apparatus is arranged to be manipulated between configurations whilst the primary chamber is isolated from the exterior of the apparatus in a fluid tight manner by the closure member.

17. Apparatus according to claim 1, wherein the apparatus is arranged to permit manipulation thereof through a plurality of configurations in a predefined order and inhibit manipulation thereof through the plurality of configurations in any other order.

18. The use of the primary chamber of apparatus according to claim 1 as both a chamber for receiving at least some of the fluid sample and testing at least some of the fluid sample.

19. A system for testing the quality of a fluid sample including:
an electronic diagnostic device; and
apparatus for testing the quality of a fluid sample according to claim 1,
the electronic diagnostic device being arranged to receive an input corresponding to the state of fluid contained within the plurality of chambers and generate an output indicative to the quality of the fluid sample, wherein the system includes a light source arranged to illuminate the chambers and the electronic diagnostic device is an optical reader arranged to measure the change in response of fluid contained in the chambers to generate the input.
